(19) United States
(12) Patent Application Publication
(10) Pub. No.: US 2012/0238804 A1
(43) Pub. Date: Sep. 20, 2012

(54) PROPELLING DEVICE AND SELF-PROPELLABLE ENDOSCOPE

(76) Inventors: Shinichi YAMAKAWA, Kanagawa (JP); Tsuyoshi Ashida, Kanagawa (JP); Takayuki Nakamura, Kanagawa (JP); Yasunori Ohta, Kanagawa (JP); Rick Cornelius, Minnesota, MN (US)

(21) Appl. No.: 13/283,693

(22) Filed: Oct. 28, 2011

Related U.S. Application Data
(60) Provisional application No. 61/453,365, filed on Mar. 16, 2011.

(51) Int. Cl. A61B 1/00

(52) U.S. Cl. 600/101

(57) ABSTRACT

A propelling device includes a rotary body, an external cylinder for supporting a rotary body in a circulating manner, a first drive mechanism, and a second drive mechanism. The first drive mechanism has a torque wire and a pinion gear that are provided in a distal portion of the insertion part. The second drive mechanism has an internal cylinder mounted on the distal portion of the insertion part, a transmission gear rotatably supported outside the internal cylinder, and a housing cylinder provided outside the transmission gear. The transmission gear has a worm gear on the outer peripheral surface thereof, and a gear tooth portion that meshes with a pinion gear on an inner peripheral surface thereof. The housing cylinder has drive gears that mesh with the worm gear. Although the second mechanism is replaced at each inspection, the first mechanism provided at the insertion part is repeatedly used.

### BACKGROUND OF THE INVENTION

1. Field of the Invention

The present invention relates to a propelling device that propels an insertion part of an endoscope within a subject, and a self-propellable endoscope equipped with the propelling device.

2. Description of the Related Art

In a medical field, an insertion part of an endoscope is inserted into a body cavity as a subject, such as a curved alimentary canal including the large intestine or the small intestine, and observation, diagnosis, and medical treatment of an inner wall surface of the alimentary canal is performed (for example, refer to JP-A-2005-253892). In particular, since the sigmoid colon of the large intestine is curved intricately and moves relatively freely, a procedure requires a level of skill to advance the insertion part to the interior within the sigmoid colon. For this reason, an endoscope which can easily advance the insertion part to the interior even within an alimentary canal such as the sigmoid colon has been needed.

In recent years, a propelling device, which is attached to a distal portion of an insertion part and propels this insertion part within the alimentary canal has been developed (for example, refer to JP-T-2009-513250). In this propelling device, a rotary body is attached to a tubular external cylinder mounted on an insertion part of an endoscope in a circulating manner, and the rotary body is circulated in a state where the outside thereof is brought into contact with the inner wall of the alimentary canal, whereby the distal portion of the insertion part is self-propellable by the friction produced between the outside of the rotary body and the inner wall of the alimentary canal.

A drive mechanism that circulates the rotary body is provided inside the external cylinder at the outer periphery of the insertion part. The drive mechanism is equipped with an internal cylinder that is mounted on the outer periphery of the insertion part of the endoscope, a cylindrical worm gear that is rotatably attached to the outside of the internal cylinder, a housing cylinder that is provided outside the worm gear, drive gears that are rotatably held by the housing cylinder, and that mesh with the worm gear and come into contact with the rotary body, and a drive source that rotates the worm gear. The worm gear is rotated by the drive source, and the drive gears are rotated by driving force received from the worm gear, so that the rotary body can be circulated to self-propel the insertion part.

When the propelling device of JP-T-2009-513250 is mounted on the distal portion of the insertion part, the apparent external diameter of the distal portion increases. Therefore, the burden on a patient who undergoes endoscopy increases. For this reason, although it is desired to make the diameter of the propelling device as small as possible, the propelling device is configured such that the drive mechanism is arranged inside the external cylinder and the rotary body, and therefore there is a problem in that it is difficult to make the diameter small.

Additionally, although the propelling device is an expendable item to be used for only one inspection, the propelling device is composed of parts of a large number, such as the external cylinder, the rotary body, and the drive mechanism. Therefore, the manufacturing cost of the propelling device becomes high. For this reason, a problem occurs in that the cost of endoscopy using the propelling device becomes high.

### SUMMARY OF THE INVENTION

An object of the present invention is to provide a propelling device that realizes a reduced diameter and low cost, and a self-propellable endoscope equipped with the propelling device.

In order to achieve the above object, a propelling device of the present invention includes a first drive mechanism and a second drive mechanism. The first drive mechanism is incorporated into an insertion part of an endoscope, and receives driving force from an external drive source. The second drive mechanism is detachably mounted on the insertion part, and generates propulsive force that propels the insertion part within a canal of a subject by the driving force received from the first drive mechanism.

The second drive mechanism preferably has a rotary body that rotates around an axis of the insertion part by the driving force received from the first drive mechanism in a state where the second drive mechanism comes into contact with an inner wall surface in the canal.

The second drive mechanism preferably has an external cylinder that allows the insertion part to be inserted therethrough, and extends along the axis of the insertion part. Preferably, the rotary body is wound around the external cylinder and is supported by the external cylinder so as to circulate along the axis.

Preferably, the propelling device further includes a plurality of supporting rollers that are rotatably attached to the external cylinder, and come into contact with an inner peripheral surface of the rotary body, so as to support the rotary body in a circulating manner. The rotary body drive gear drives the rotary body in a state where the rotary body is pinched between the rotary body drive gear and the plurality of supporting rollers. The rotary body is preferably formed in the shape of a bag so as to cover the external cylinder over its entire circumference.

According to an embodiment of the present invention, the first drive mechanism has a driving-force transmission gear and a torque wire. The driving-force transmission gear is provided inside the insertion part, and has a rotating shaft parallel to the axis, and partially protrudes from an opening formed at the outer periphery of the inserting part. The torque wire is inserted into the inside of the insertion part to transmit the power from the drive source to the rotating shaft, thereby rotating the driving-force transmission gear. The second drive mechanism has an internal cylinder, a worm gear, and a rotary body drive gear. The internal cylinder is detachably mounted on the outer periphery of the insertion part, and has an opening for exposing the partially protruded driving-force transmission gear. The worm gear is rotatably attached to the outside of the internal cylinder, has a gear tooth portion that meshes with the driving-force transmission gear on the inner peripheral surface thereof, and is rotated by driving force received from the driving-force transmission gear. The rotary body drive gear is provided outside the worm gear, and rotates the rotary body by the driving force received from the worm gear.

Preferably, the second drive mechanism has a housing cylinder. The housing cylinder is detachably provided outside the worm gear to house the worm gear, and rotatably holds the rotary body drive gear.

According to another embodiment of the present invention, the first drive mechanism has an internal cylinder, a worm gear, a worm gear rotation driving device, and a rotary body drive gear. The internal cylinder is provided inside the insertion part. The worm gear is rotatably attached to the outside of the internal cylinder, and at least partially exposed from the opening for a worm gear provided at the outer periphery of the insertion part. The worm gear rotation driving device rotates the worm gear in the circumferential direction thereof by the driving force received from the drive source. The rotary body drive gear is rotatably attached to the opening for a worm gear, and rotates the rotary body by the driving force received from the worm gear.

According to further another embodiment of the present invention, the first drive mechanism has an internal cylinder, a worm gear, and worm gear rotation driving device, and the second drive mechanism has a rotary body drive gear and a gear holding element.

The rotary body drive gear rotates the rotary body by the driving force received from the worm gear with which the rotary body drive gear meshes via the opening for a worm gear. The gear holding element is detachably installed outside the insertion part and rotatably holds the rotary body drive gear.

A gear tooth portion is formed along the circumferential direction of an inner periphery of the worm gear. Preferably, the worm gear rotation driving device has a driving-force transmission gear that has a rotating shaft parallel to the axis and meshes with the gear tooth portion, and a torque wire that transmits the power received from the drive source to the rotating shaft of the driving-force transmission gear, thereby rotating the driving-force transmission gear.

Additionally, a self-propellable endoscope of the present invention includes an insertion part inserted into a canal of a subject, an operation part for operating the insertion part, and the propelling device described above.

According to the present invention, since the second drive mechanism detachably mounted on the insertion part receives driving force from the first drive mechanism incorporated into the insertion part, and generates propulsive force, the diameter of the propelling device can be made small by an amount equivalent to the first drive mechanism incorporated into an empty space or the like in the insertion part. Additionally, it becomes unnecessary to replace all the parts of the propelling device at every endoscopy differently from the conventional endoscopy, and at least the first drive mechanism can be used repeatedly. Thereby, since the cost of the propelling device required for each inspection is suppressed lower than before, the cost of endoscopy can be decreased.

### BRIEF DESCRIPTION OF THE DRAWINGS

The above object and advantages can be easily understood by those skilled in the art by reading the detailed description of the preferred embodiments of the invention with reference to the attached drawings:

FIG. 1 is a schematic view of a self-propellable endoscope;

FIG. 2 is a perspective view of a propelling device;

FIG. 3 is an exploded perspective view of the propelling device;

FIG. 4 is a cross-sectional view when the propelling device is seen from the front;

FIG. 5 is a cross-sectional view when the propelling device is seen from the side;

FIG. 6 is a perspective view of a distal portion of an insertion part;

FIG. 7 is an expanded cross-sectional view showing the section of the distal portion of the insertion part in an enlarged manner;

FIG. 8 is a cross-sectional view of a propelling device of a second embodiment; and

FIG. 9 is a cross-sectional view of a propelling device of a third embodiment.

### DESCRIPTION OF THE PREFERRED EMBODIMENTS

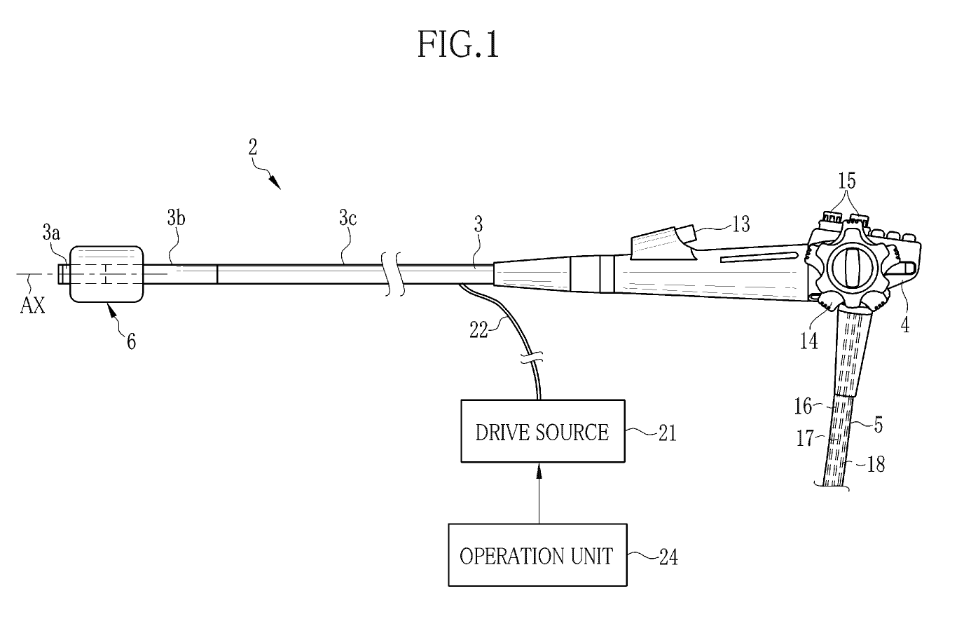

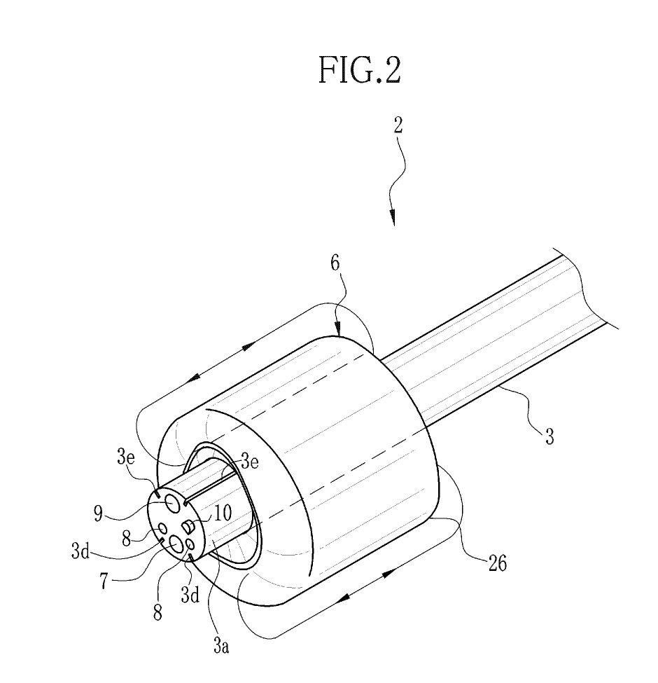

As shown in FIGS. 1 and 2, a self-propellable endoscope (hereinafter simply referred to as an endoscope) 2 is constituted by an insertion part 3, an operating part 4, a universal cord 5, and a propelling device 6. The insertion part 3 has a CCD type or CMOS type image sensor (not shown) built therein, and is inserted into alimentary canals such as the large intestine as a subject. The operating part 4 is used for the grip of the endoscope 2 and the operation of the insertion part 3. The universal cord 5 is used for connecting the endoscope 2 to a processor, alight source unit, and air/water sending device (none of them are illustrated). The propelling device 6 propels the insertion part 3 within the alimentary canal.

The insertion part 3 is composed of a hard distal portion 3a having an image sensor built therein, a curved portion 3b connected to a rear end of the distal portion 3a and capable of being curved in the up-and-down direction and in the right-and-left direction, and a flexible portion 3c connected to a rear end of the curved portion 3b and having flexibility. In addition, a symbol AX represents an axis (central line) of the insertion part 3.

The distal portion 3a is provided with an observation window 7 arranged in front of the image sensor, an illumination window 8 for emitting illumination light from the light source unit, a forceps outlet 9 as an outlet of a forceps channel (not shown) inserted through the insertion part 3, and an injection nozzle 10 for injecting air or cleaning water toward the observation window 7.

The operating part 4 is provided with a forceps inlet 13 which communicates with the forceps channel, an angle knob 14 for curving the curved portion 3b in the up-and-down direction and in the right-and-left direction, and operation buttons 15 used during various operations such as air sending, water sending, and suction.

The universal cord 5 is connected to the operating part 4. An air/water sending tube 16, an imaging signal outputting cable 17, and a light guide 18 are incorporated into the universal cord 5. The air/water sending tube 16 has one end connected to the air/water sending device and the other end connected to the injection nozzle 10, and sends air or cleaning water supplied from the air/water sending device to the injection nozzle 10. The imaging signal outputting cable 17 has one end connected to the processor and the other end connected to the image sensor. The light guide 18 has one end connected to the illumination window 8 and the other end connected to the light source unit, and guides the illumination light radiated from the light source unit to the illumination window 8.

The propelling device 6 is provided from the distal portion 3a to the curved portion 3b, and advances or retreats the insertion part 3 within the alimentary canal. In addition, the position on the insertion part 3 where the propelling device 6 is provided may be changed appropriately. The propelling device 6 is driven, for example, by a drive source 21 such as a motor. The drive source 21 generates rotary torque for propelling the propelling device 6, and transmits the rotary torque to the propelling device 6 via a torque wire 22 coupled to the drive source 21.

The torque wire 22 is inserted through the inside of a protective sheath made of resin, for example, and is turned within the protective sheath by the driving of the drive source 21. The torque wire 22 is inserted into the insertion part 3.

An operation unit 24 is connected to the drive source 21. The operation unit 24 is equipped with buttons for inputting instructions to advance, retreat, and stop of the propelling device 6, a speed adjustment button for adjusting the movement speed of the propelling device 6, and the like.

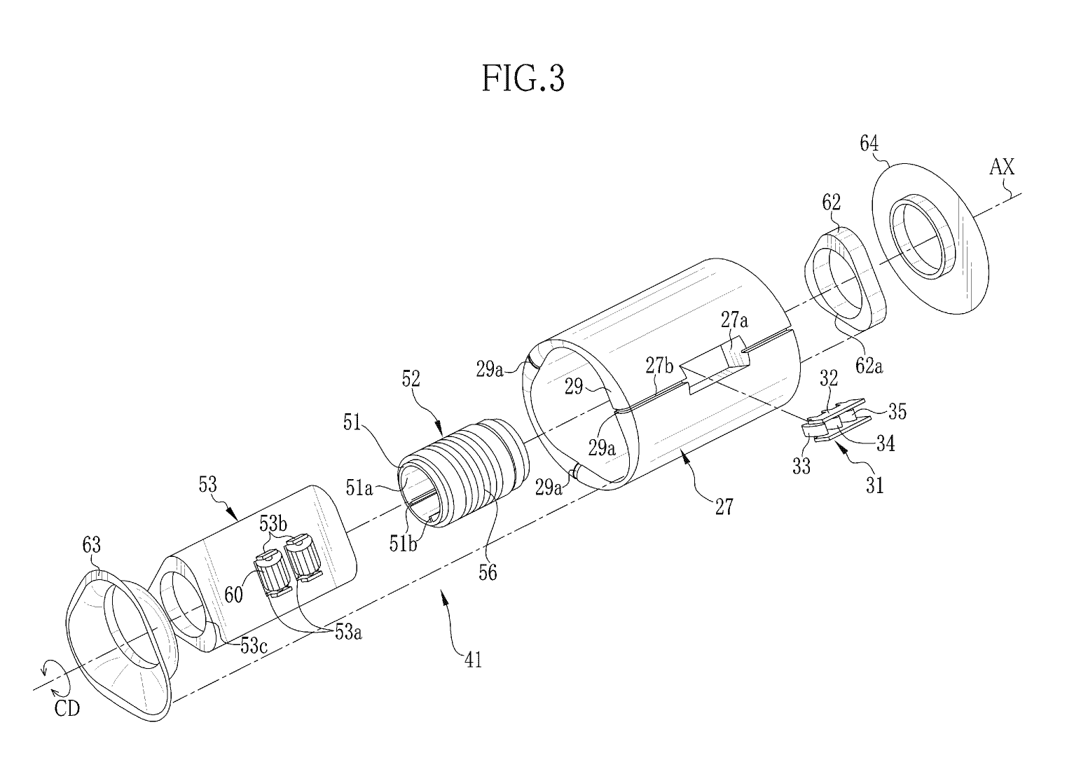

In FIG. 2, the propelling device 6 comes into contact with the inner wall surface of the alimentary canal or the like, and is equipped with a rotary body (also referred to as a toroid) 26 for producing propulsive force in an extraction direction opposite to an insertion direction of the insertion part 3 of the endoscope 2. The rotary body 26 is supported by an external cylinder 27 (refer to FIG. 3), so as to move along an axis AX in a circulating manner, and covers the external cylinder 27 over its entire circumference. Note that, the arrow in the drawing indicates the direction of the circulation of the rotary body 26. The rotary body 26 is formed from, for example, biocompatible plastics, such as polyvinyl chloride, polyamide resin, fluororesin, urethane, and polyurethane, and has flexibility.

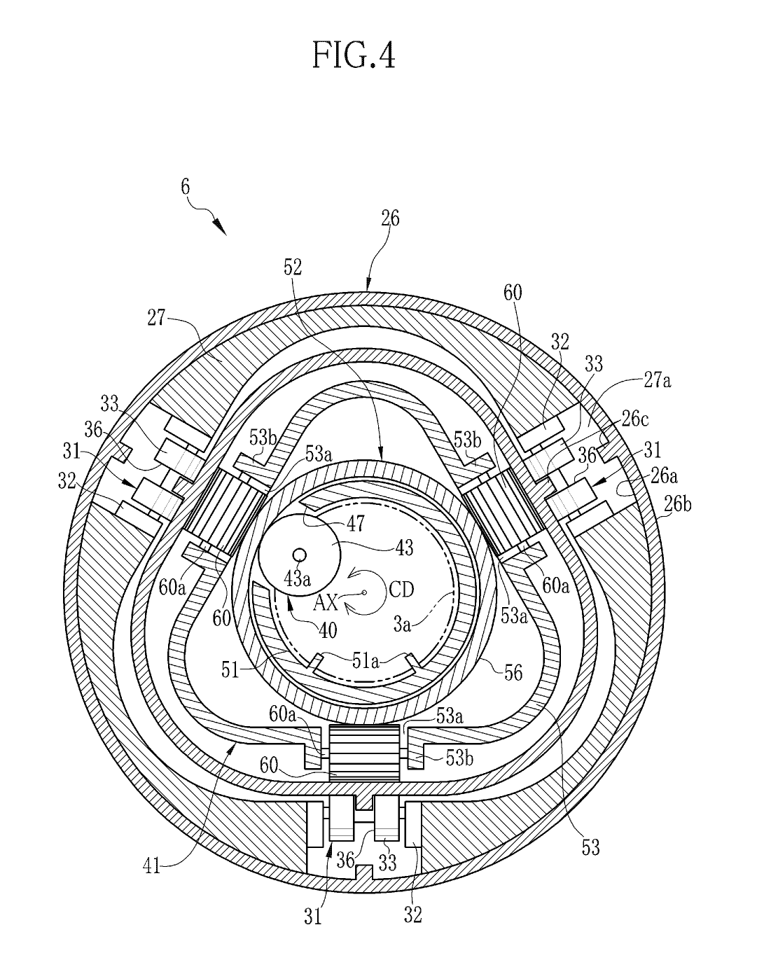

As shown in FIGS. 3 to 5, the external cylinder 27 is a tubular body, and the cross-section of the tubular body in a direction orthogonal to the axis AX has a circular shape on the outer peripheral surface, and has a substantially triangular shape (corresponding to a shape in which each angle of an equilateral triangle is curved and rounded) on the inner peripheral surface. The rotary body 26 is wounded around the external cylinder 27. Note that, illustration of the rotary body 26 is omitted in FIG. 3.

The rotary body 26 is formed in a cylindrical shape at first, and passed through the external cylinder 27. Then, both ends of the rotary body 26 are folded outward so as to be overlapped with each other, and the both ends are bonded together into an endless state by thermal welding or the like.

A ring-shaped contact body 29 that comes into contact with the rotary body 26 is attached to each of front and rear ends of the external cylinder 27. The contact body 29 is made of materials allowing the rotary body 26 to circulate smoothly, such as nylon, PEEK, and Teflon.

Three straight-line portions are provided on the inner peripheral surface of the external cylinder 27, and each straight-line portion is formed with an opening 27a for a roller. A roller unit 31 for supporting the rotary body 26 in a circulating manner is attached to each of the openings 27a. In the roller unit 31, first to third supporting rollers 33 to 35 are rotatably attached in order along the axis AX between two supporting plates 32. In addition, the respective supporting rollers 33 to 35 may be rotatably attached to the external cylinder 27 itself. Additionally, the locations where the roller units 31 are attached are not limited to three, and the number of the roller units may be appropriately changed.

An inner surface 26a of the rotary body 26 comes into contact with the respective supporting rollers 33 to 35. The portions of the rotary body 26, which come into contact with the respective supporting rollers 33 to 35, are made thicker than other portions thereof, and thereby have high rigidity. Note that, reference numeral 26b designates an outer surface of the rotary body 26.

A groove portion 36 is formed at a central portion of each of the supporting rollers 33 to 35. Three linear projections 26c are formed on the inner surface 26a of the rotary body 26. The linear projection 26c is formed over its entire circumference. The linear projection 26c is slidably engaged with the groove portion 36, and prevents the rotary body 26 from rotating in a circumferential direction CD. Similarly, the external cylinder 27 is formed with a groove portion 27b with which the linear projection 26c is slidably engaged, and the contact body 29 is formed with a groove portion 29a with which the linear projection 26c is slidably engaged. In addition, lubricant is applied between the groove portion 27b and the linear projection 26c, between the groove portion 29a and the linear projection 26c, and between the groove portion 36 and the linear projection 26c in order to enhance the slidability therebetween, respectively.

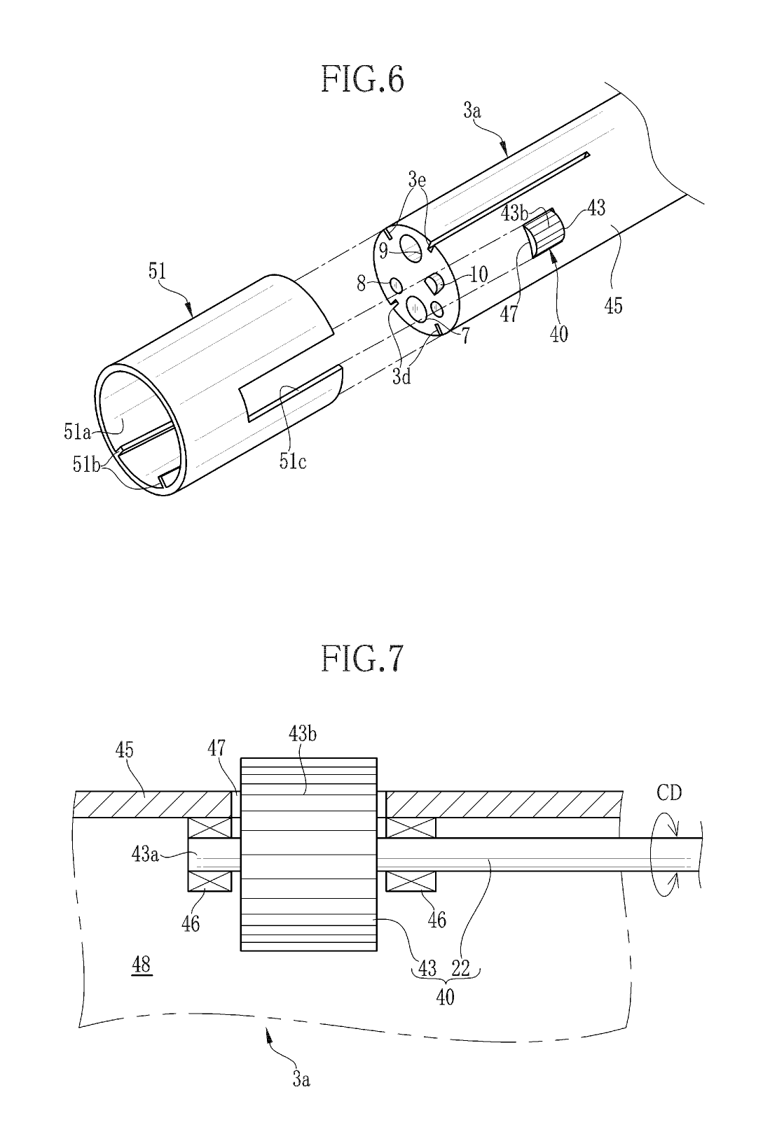

The propelling device 6 is provided with a drive mechanism that generates propulsive force for propelling the insertion part 3 within the alimentary canal or the like. The drive mechanism is composed of a first drive mechanism 40 (refer to FIGS. 6 and 7) incorporated into the insertion part 3, and a second drive mechanism 41 that is detachably mounted on the insertion part 3.

As shown in FIGS. 6 and 7, the first drive mechanism 40 is composed of the torque wire 22 and a pinion gear (driving-force transmission gear) 43. The pinion gear 43 has a rotating shaft 43a parallel to the axis AX. The rotating shaft 43a is rotatably held by bearings 46 provided in the inner peripheral surface of a tubular outer peripheral portion 45 constituting the distal portion 3a. Additionally, one part of an outer peripheral portion of the pinion gear 43 having gear teeth 43b protrudes from an opening 47 for a pinion gear which is formed at the outer peripheral portion 45 of the distal portion 3a, and the other part thereof is housed in an internal space 48 of the insertion part 3.

A distal portion of the torque wire 22 is coupled to the rotating shaft 43a. Thereby, when the rotary torque from the drive source 21 is transmitted to the rotating shaft 43a via the torque wire 22, the pinion gear 43 rotates about the rotating shaft 43a.

Referring to FIGS. 3 to 5, the second drive mechanism 41 is composed of a cylindrical internal cylinder 51 that is detachably mounted on the distal portion 3a, a transmission gear 52 that is rotatably supported outside the internal cylinder 51, a housing cylinder 53 that houses the internal cylinder 51 and the transmission gear 52 so as to become coaxial with them, the rotary body 26, and the external cylinder 27. In addition, the transmission gear 52, the housing cylinder 53, the rotary body 26, and the external cylinder 27 also can be detached from the distal portion 3a.

The inner peripheral surface of the internal cylinder 51 is formed with an insertion hole 51a through which the distal portion 3a and the curved portion 3b are inserted, and two positioning ribs 51b for positioning the circumferential direction of the distal portion 3a. Additionally, as shown in FIG. 2, the outer peripheral surfaces of the distal portion 3a and the curved portion 3b are formed with observation window positioning recesses 3d for disposing the observation window 7 at the radial center of the propelling device 6, and forceps outlet positioning recesses 3e for disposing the forceps outlet 9 at the center. The positioning ribs 51b are inserted into the observation window positioning recesses 3d or the forceps outlet positioning recesses 3e.

The internal cylinder 51 is formed with an opening 51c for a pinion gear for exposing the pinion gear 43 partially protruding from the opening 47 of the distal portion 3a. As shown in FIG. 6, a rear end of the opening 51c becomes a guide port for allowing the pinion gear 43 to pass therethrough when the internal cylinder 51 is mounted.

The transmission gear 52 is formed in a cylindrical shape, and externally fitted to the internal cylinder 51, so as to rotate about the axis AX. The transmission gear 52 has a spiral worm gear 56 and a gear tooth portion 57. The worm gear 56 is formed on the outer peripheral surface of the transmission gear 52 with its center at the axis AX. The gear tooth portion 57 is formed on the inner peripheral surface of the transmission gear 52, and has a plurality of gear teeth arrayed in the circumferential direction thereof. The axial position of the gear tooth portion 57 along the axis AX coincides with that of each of the openings 47 and 51c, and the gear tooth portion 57 meshes with the pinion gear 43. Thereby, when the pinion gear 43 rotates, the gear tooth portion 57 rotates such that the transmission gear 52 also rotates in the circumferential direction.

The housing cylinder 53 is formed in a substantially triangular tubular shape (corresponding to a shape in which each angle of an equilateral triangle is curved and rounded), and is disposed so as to have the same axial position as that of the external cylinder 27. An Opening 53a is formed in each of three straight-line portions of the housing cylinder 53. Two gears 60 for driving a rotary body (hereinafter simply referred to as drive gears 60) are disposed in each of the openings 53a. Each of the drive gears 60 has a rotating shaft 60a substantially perpendicular to the axis AX, and is rotatably attached to an attachment rib 53b formed on the housing cylinder 53. The drive gear 60 is disposed between the first supporting roller 33 and the second supporting roller 34 and between the second supporting roller 34 and the third supporting roller 35, respectively.

The respective drive gears 60 mesh with the worm gear 56 of the transmission gear 52, and come into contact with the outer surface 26b of the rotary body 26, such that the rotary body 26 is pinched between the worm gear 56 and the first to third supporting rollers 33 to 35. Each of the drive gears 60 overlaps with each of the supporting rollers 33 to 35 in the radial direction of the external cylinder 27, and the rotary body 26 is curved in a wavelike fashion between each of the supporting rollers 33 to 35 and each of the drive gears 60. Thereby, when the worm gear 56 rotates in the circumferential direction, each of the drive gears 60 rotates and the rotary body 26 is circulated.

The front surface of the housing cylinder 53 is formed with an opening 53c. A distal portion of the internal cylinder 51 is inserted into the opening 53c. A lid 62 is attached to a rear end of the housing cylinder 53. A front stopper 63 that prevents entering of the inner wall of the alimentary canal is attached to the tip of the housing cylinder 53, and a rear stopper 64 is attached to the lid 62, similarly.

The lid 62 is formed in the same shape as that of the housing cylinder 53 (namely, in a substantially triangular shape), and has an opening 62a that communicates with the insertion hole 51a of the internal cylinder 51. The front stopper 63 and the rear stopper 64 are respectively formed in a shape like a mortar so as to block a gap formed between the external cylinder 27 and the internal cylinder 51, and prevent the inner wall of the alimentary canal from entering inside of the propelling device 6 in accordance with the circulation of the rotary body 26.

Next, an operation of the propelling device 6 will be described. First, the distal portion 3a of the endoscope 2 is fitted into the insertion hole 51a of the internal cylinder 51, and the propelling device 6 is mounted on the distal portion 3a. At this time, for example, the positioning ribs 51b are inserted into the positioning recesses 3d for an observation window, such that the observation window 7 is disposed at the center of the propelling device 6. Next, a power source for each of the processor, the light source unit, the operation unit 24, and the like is turned on to perform inspection preparation. After the inspection preparation is completed, the insertion part 3 of the endoscope 2 is inserted into a patient's alimentary canal, for example, large intestine.

When the operation unit 24 is operated and an advance instruction is input after the distal portion 3a is advanced up to a predetermined position in the large intestine, for example, just before the sigmoid colon, rotary torque is generated from the drive source 21, the torque wire 22 is rotated in a predetermined direction, and the pinion gear 43 is further rotated in the same direction via the torque wire 22. Thereby, the gear tooth portion 57 that meshes with the pinion gear 43 rotates, and the transmission gear 52 rotates.

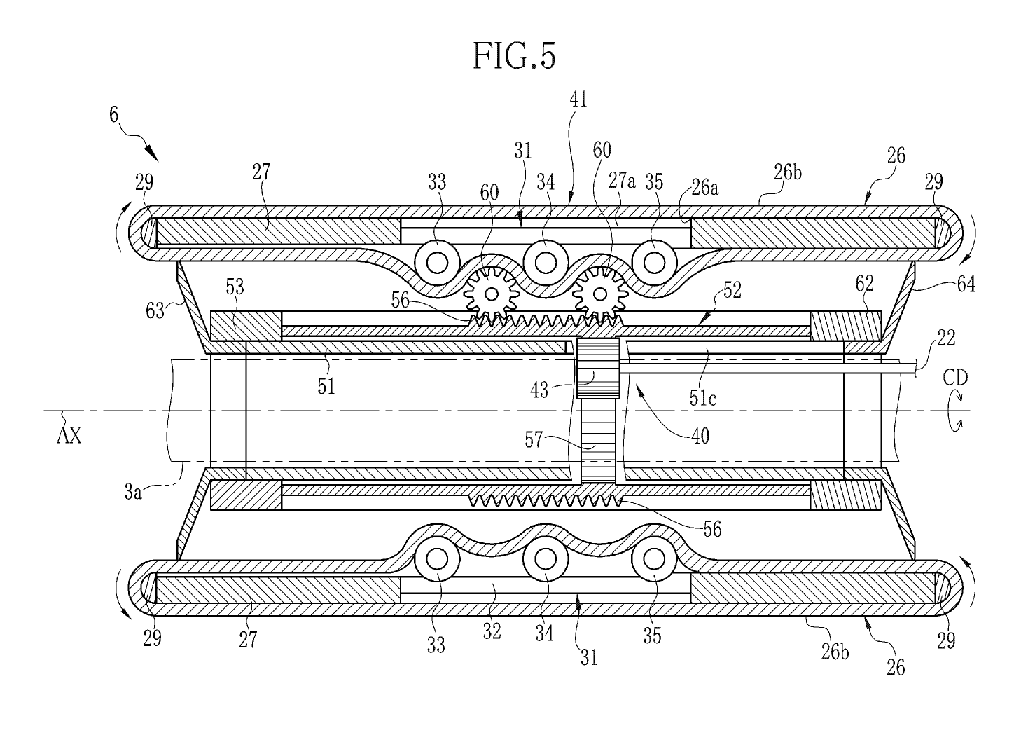

Since the worm gear 56 rotates in the circumferential direction in accordance with the rotation of the transmission gear 52, each of the drive gears 60 that meshes with the worm gear 56 rotates. In accordance with the rotation of each of the drive gears 60, the rotary body 26 pinched between each of the drive gears 60 and each of the supporting rollers 33 to 35 rotates in a direction indicated by the arrow of FIG. 5. At this time, the outer surface 26b of the rotary body 26 that comes into contact with the inner wall of the large intestine outside the external cylinder 27 moves in the extraction direction opposite to the insertion direction. Additionally, the outer surface 26b of the rotary body 26 located inside the external cylinder 27 simultaneously moves in the insertion direction. Thereby, the rotary body 26 moves in a circulating manner.

Since the rotary body 26 comes into contact with the inner wall of the large intestine, force for pulling the inner wall of the large intestine from the front of the insertion part 3 to the rear thereof is generated by the circular movement. Thereby, the distal portion 3a advances along the inner wall of the large intestine. On the other hand, when the propelling device 6 is retreated in the extraction direction, the rotary body 26 circulates in the direction reverse to the above.

When a speed change instruction is input to the operation unit 24, the rotating speed of the torque wire 22 to be caused by the drive source 21 is changed, and the movement speed of the propelling device 6 is changed. Additionally, when a retreat instruction is input to the operation unit 24, the torque wire 22 is reversely rotated and the propelling device 6 retreats. Moreover, when a stop instruction is input to the operation unit 24, the driving of the drive source 21 stops, the rotation of the torque wire 22 is stopped, and the propelling device 6 stops. By appropriately performing the above operations, the distal portion 3a can be moved to a desired position in the large intestine.

In this case, in the propelling device 6, the first drive mechanism 40 composed of the torque wire 22 and the pinion gear 43 is incorporated into the insertion part 3, and therefore a diameter can be made smaller by a dimension equivalent to the first drive mechanism 40 than a conventional propelling device (refer to JP-T-2009-513250) in which the first drive mechanism 40 is provided outside the insertion part 3. In addition, since the first drive mechanism 40 can be arranged in an empty space such as the internal space 48 in the insertion part 3, even if the first drive mechanism 40 is provided in the insertion part 3, an increase in the diameter of the insertion part 3 can be prevented. Thereby, since an increase in the apparent external diameter of the distal portion 3a is prevented, the burden on a patient who undergoes endoscopy can be reduced.

Additionally, since the first drive mechanism 40 is isolated from the inside of the large intestine by the internal cylinder 51, the transmission gear 52, and the front and rear stoppers 63 and 64, the first drive mechanism 40 is not contaminated during endoscopy. For this reason, in the propelling device 6, it is necessary to replace the second drive mechanism 41, the front and rear stoppers 63 and 64, and the like at each inspection, but the first drive mechanism 40 can be used repeatedly. As a result, since the cost of the propelling device 6 that is required for each inspection is suppressed to be lower than before, the cost of endoscopy can be decreased.

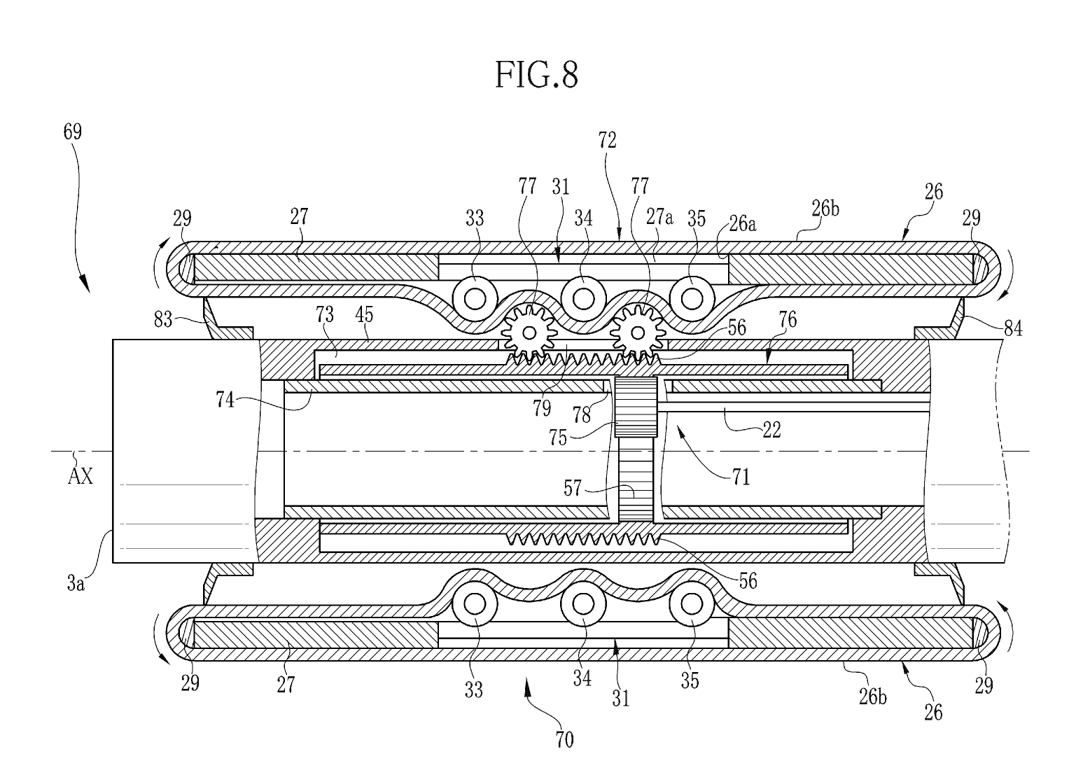

Next, an endoscope 69 of a second embodiment of the present invention will be described with reference to FIG. 8. In the endoscope 2 of the above first embodiment, the first drive mechanism 40 composed of the torque wire 22 and the pinion gear 43 is incorporated into the insertion part 3. However, according to the second embodiment, the number of parts to be incorporated into the insertion part 3 is increased in comparison with that of the first embodiment.

Except that the endoscope 69 has a propelling device 70 different from the propelling device 60 of the first embodiment, and that the outer peripheral portion 45 of the distal portion 3a is faced to the rotary body 26, the endoscope 69 basically has the same configuration as the endoscope 2 of the first embodiment. The same components as those of the above first embodiment in terms of functions and structure are designated by the same reference numerals, and the description thereof is omitted. Additionally, except that the propelling device 70 is equipped with a first drive mechanism 71 and a second drive mechanism 72 that are respectively different from the first drive mechanism 40 and the second drive mechanism 41 of the first embodiment, the propelling device 70 basically has the same configuration as that of the propelling device 6 of the first embodiment. The second drive mechanism 72 is composed of the rotary body 26 and the external cylinder 27.

The first drive mechanism 71 is incorporated into the insertion part 3. The first drive mechanism 71 is composed of a cylindrical internal cylinder 74 that is disposed in an internal space 73 of the insertion part 3, the torque wire 22 and a pinion gear 75 that are provided inside the internal cylinder 74, a transmission gear 76 that is rotatably supported outside the internal cylinder 74, and drive gears 77 that are attached to the outer peripheral portion 45. In addition, a worm gear rotation driving device of the present invention is constituted by the torque wire 22 and the pinion gear 75.

The inner peripheral surface of the internal cylinder 74 is provided with the same bearing (not shown) as the bearing 46 shown in FIG. 7, and this bearing rotatably supports the rotating shaft of the pinion gear 75. Additionally, the internal cylinder 74 is formed with an opening 78 for a pinion gear at a position where the pinion gear 75 is held. Thereby, one part of the outer peripheral portion of the pinion gear 75 protrudes from the opening 78 to the outside of the internal cylinder 74, and the other part thereof is housed in the internal cylinder 74. In addition, except that the pinion gear 75 is attached to the inner peripheral surface of the internal cylinder 74, the pinion gear 75 is the same as the pinion gear 43 of the first embodiment.

The transmission gear 76 is externally fitted to the internal cylinder 74 in the internal space 73, and rotates about the axis AX. The transmission gear 76, similarly to the transmission gear 52 of the first embodiment, has the worm gear 56 formed on the outer peripheral surface thereof, and the gear tooth portion 57 formed on the inner peripheral surface thereof. The axial position of the gear tooth portion 57 coincides with that of the opening 78, and the gear tooth portion 57 meshes with the pinion gear 75 protruding from the opening 78. Thereby, in accordance with the rotation of the pinion gear 75, the gear tooth portion 57 and the transmission gear 76 rotate in the circumferential direction.

The worm gear 56 is partially exposed from an opening 79 for a worm gear which is formed on the outer peripheral portion 45. A peripheral edge of the opening 79 is provided with an attachment rib (illustration thereof is omitted) that rotatably holds a rotating shaft of the drive gear 77 in a posture substantially perpendicular to the axis AX. The attachment rib is basically the same as the attachment rib 53b shown in FIG. 3.

Except that the drive gear 77 is attached to the outer periphery of the distal portion 3a, the drive gear 77 is basically the same as the drive gear 60 of the first embodiment, and is disposed between the first and second supporting rollers 33 and 34, and between the second and third supporting rollers 34 and 35, respectively. The respective drive gears 77 mesh with the worm gear 56 and come into contact with the outer surface 26b of the rotary body 26, so as to pinch the rotary body 26 between the drivers 77 and the first to third supporting rollers 33 to 35. Thereby, when the worm gear 56 rotates in the circumferential direction, each of the drive gears 77 rotates, and the rotary body 26 is circulated.

The outer periphery of the distal portion 3a or the like is provided with a front stopper 83 and a rear stopper 84 that prevent entering of the inner wall of the alimentary canal into a gap formed between the outer periphery and the external cylinder 27.

Next, the operation of the propelling device 70 of the second embodiment will be described. Similarly to the first embodiment, after the propelling device 70 is mounted on the distal portion 3a, and a power source for each of the processor, the light source unit, the operation unit 24, and the like is turned on to perform inspection preparation, the insertion part 3 is inserted into a patient's alimentary canal such as large intestine.

When the operation unit 24 is operated and an advance instruction is input after the distal portion 3a is advanced, for example, just before the sigmoid colon, a rotary torque is generated from the drive source 21, the torque wire 22 is rotated in a predetermined direction, and the pinion gear 75 is further rotated in the same direction via the torque wire 22. Thereby, the gear tooth portion 57 rotates, and the transmission gear 76 rotates.

Since the worm gear 56 rotates in the circumferential direction in accordance with the rotation of the transmission gear 76, each of the drive gears 77 rotates. Thereby, similarly to the first embodiment, the distal portion 3a advances along the inner wall of the large intestine as the rotary body 26 rotates in a direction indicated by the arrow of FIG. 8.

Hereinafter, similarly to the first embodiment, when a speed change instruction, a retreat instruction, and a stop instruction are input to the operation unit 24, the movement speed of the propelling device 70 changes, and the retreating and stopping of the propelling device 70 are executed, respectively. Thereby, the distal portion 3a of the endoscope 69 can be moved to a desired position in the large intestine.

In this case, in the propelling device 70, the first drive mechanism 71 composed of the torque wire 22, the internal cylinder 74, the pinion gear 75, the transmission gear 76, and the drive gear 77 is incorporated into the insertion part 3. Thus, the number of parts to be incorporated into the insertion part 3 is increased in comparison with the propelling device 6 of the first embodiment. For this reason, the diameter of the propelling device 70 can be made much smaller than the propelling device 6 of the first embodiment. In addition, similarly to the first embodiment, since the first drive mechanism 71 can be arranged in an empty space in the insertion parts 3, such as the internal space 73, an increase in the diameter of the insertion part 3 can be prevented.

Additionally, in the propelling device 70, parts other than the second drive mechanism 72 composed of the rotary body 26 and the external cylinder 27, and the front and rear stoppers 83 and 84 are incorporated into the distal portion 3a. The endoscope 69 is usually subjected to cleaning disinfection treatment after endoscopy. At this time, the respective parts mounted on the endoscope 69 are also subjected to cleaning disinfection treatment. For this reason, it is necessary to replace the rotary body 26, the external cylinder 27, the front and rear stoppers 83 and 84, and the like at each inspection, but the parts other than those can be used repeatedly. Since the number of parts that can be used only once decreases in comparison with the first embodiment, the cost of the propelling device 70 taken for each inspection can be suppressed lower, and the cost of endoscopy can be decreased, in comparison with the first embodiment.

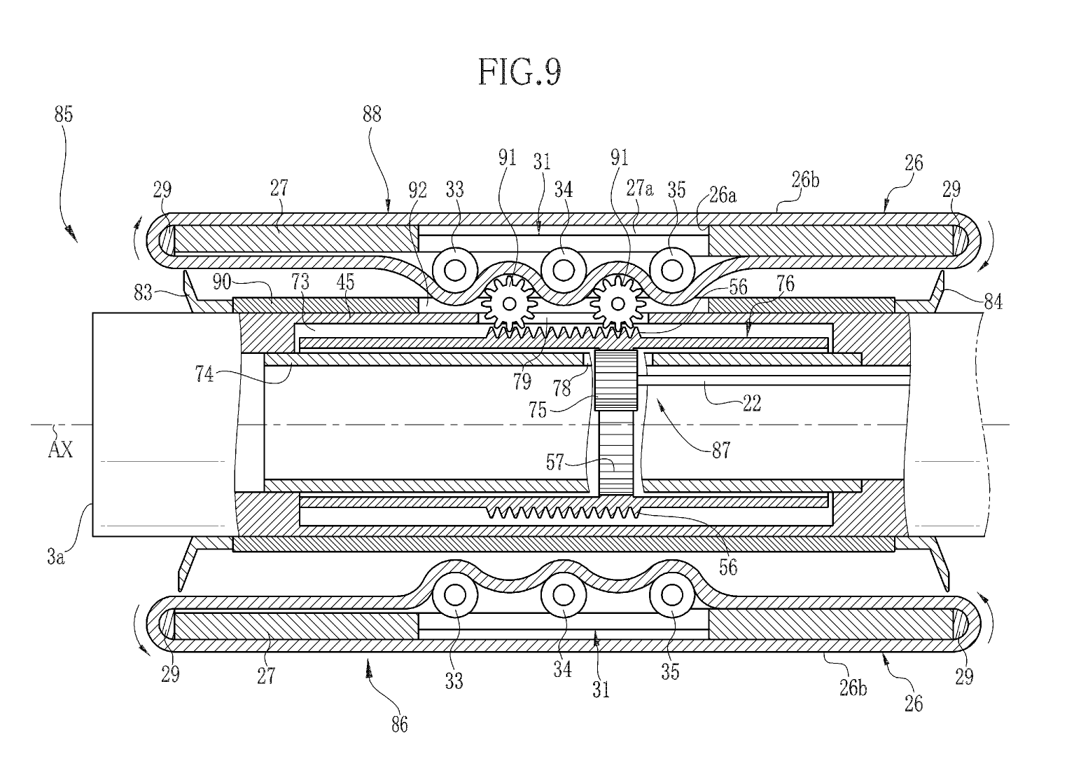

Next, an endoscope 85 of a second embodiment of the present invention will be described with reference to FIG. 9. In the endoscope 85, the number of parts to be incorporated into the insertion part 3 is increased in comparison with that of the endoscope 2 of the first embodiment. However, the number of parts to be incorporated into the insertion part 3 is smaller than that of the endoscope 69 of the second embodiment.

Except that the endoscope 85 has a propelling device 86 different from the propelling devices 6 and 70 of the first and second embodiments, and that the drive gears are detachably and rotatably held on the outer peripheral portion 45, the endoscope 85 basically has the same configuration as those of the endoscopes 2 and 69 of the first and second embodiments. The same components as those of the above first and second embodiments in terms of functions and structure are designated by the same reference numerals, and the description thereof is omitted.

The first drive mechanism 87 is incorporated into the internal space 73 of the insertion part 3. The first drive mechanism 87 is composed of the internal cylinder 74, the torque wire 22, the pinion gear 75, and the transmission gear 76, which are the same as those of the first drive mechanism 71 of the second embodiment, and the gear tooth portion 57 of the transmission gear 76 and the transmission gear 76 rotate in the circumferential direction in accordance with the rotation of the pinion gear 75.

The second drive mechanism 88 is composed of a gear holding cylinder (gear holding element) 90 that is detachably mounted on the outside of the outer peripheral portion 45, drive gears 91 that are rotatably held by the gear holding cylinder 90, the rotary body 26, and the external cylinder 27. The gear holding cylinder 90 has an opening 92 for holding a drive gear which is formed at a position facing the opening 79 formed at the outer peripheral portion 45. Thereby, the worm gear 56 is exposed from the opening 92 via the opening 79.

Except that the drive gear 91 is attached to a rotating shaft substantially perpendicular to the axis AX provided in the opening 92 for holding a drive gear, the drive gear 91 is basically the same as the drive gears 60 and 77 of the first and second embodiments, and is disposed between the first supporting roller 33 and the second supporting roller 34, and between the second supporting roller 34 and the third supporting roller 35, respectively. The respective drive gears 91 mesh with the worm gear 56 via the opening 79 and the opening 92, and come into contact with the outer surface 26b of the rotary body 26, so as to pinch the rotary body 26 between the drive gears 91 and the supporting rollers 33 to 35. Thereby, when the worm gear 56 rotates in the circumferential direction, each of the drive gears 91 rotates, and the rotary body 26 is circulated.

Since the operation of the propelling device 86 of the third embodiment is basically the same as that of each of the propelling devices 6 and 70 of the first and second embodiments, the description thereof is omitted here. In the propelling device 86, the first drive mechanism 87 composed of the torque wire 22, the internal cylinder 74, the pinion gear 75, and the transmission gear 76 is incorporated into the insertion part 3. Thus, the number of parts to be incorporated into the insertion part 3 is made larger than that of the propelling device 6 of the first embodiment. For this reason, the diameter of the propelling device 86 can be made smaller than that of the propelling device 6 of the first embodiment, and the cost of endoscopy can be made lower than that of the first embodiment.

Additionally, according to the third embodiment, since the number of parts to be incorporated into the insertion part 3 is made smaller than that of the first drive mechanism 71 of the second embodiment, even when an empty space that can house the first drive mechanism 71 cannot be sufficiently secured in the insertion part 3, the first drive mechanism 87 may be able to be incorporated therein. Accordingly, the propelling device of any one of the first to third embodiments is selected according to the size of an empty space in the insertion part 3.

In the above third embodiment, the drive gears 91 are detachably held on the outer peripheral portion 45 by the gear holding cylinder 90. However, the drive gears 91 may be detachably held on the outer peripheral portion 45 with use of members having various shapes other than the gear holding cylinder 90.

In the above respective embodiments, the internal cylinder and the external cylinder are respectively formed in the shape of a triangular cross-section and a circular cross-section. However, in addition to the above shapes, the internal cylinder and the external cylinder may be respectively formed in the shape of a circular shape and a polygonal shape.

In the above respective embodiments, the endoscope is advanced or retreated by the rotary body 26 that covers the external cylinder 27 over its entire circumference. However, the present invention is also applicable to a propelling device that advances or retreats an endoscope by various rotary bodies, such as rollers rotatably supported by various support members, such as a plurality of endless belts that cover a part of the external cylinder 27 in the circumferential direction, or the external cylinder 27.

In the above respective embodiments, although the rotary body 26 is driven in a circulating manner by rotating the drive gears 60, 77, and 91 by the worm gear 56 of the transmission gears 52 and 76, the rotary body 26 may be directly driven by the worm gear 56. In addition, in accordance with the existence or non-existence of the drive gears, the rotational direction of the worm gear for advancing or retreating the endoscope becomes reversed. Therefore, it is necessary to change the relationship between an advance/retreat instruction made by the operation unit and the rotational direction of the torque wire caused by the drive source.

In the above respective embodiments, although the transmission gears 52 and 76 are driven using the pinion gears 43 and 75, the shape, size, and the like of a driving-force transmission gear for driving the transmission gears 52 and 76 may be arbitrarily decided. Additionally, in the second embodiment, the gear tooth portion 57 is provided on the inner peripheral surface of the transmission gear 76. However, the gear tooth portion 57 may be provided on the outer peripheral surface of the transmission gear 76, and the pinion gear 75 may be provided outside the transmission gear 76. Additionally, in the second and third embodiments, any drive mechanism may be used as a mechanism for driving the transmission gear 76 to rotate.

In the above embodiments, the present invention is applied to an endoscope for medical diagnosis. However, the present invention may be applied to other industrial endoscopes, probes, or the like.

Various changes and modifications are possible in the present invention and may be understood to be within the present invention.

What is claimed is:

1. A propelling device for propelling an insertion part of an endoscope in a subject by receiving driving force from a drive source, said propelling device comprising:
a first drive mechanism that is incorporated into said insertion part and receives the driving force from said drive source; and
a second drive mechanism that is detachably mounted on said insertion part, and receives the driving force from said first drive mechanism, so as to generate propulsive force for propelling said insertion part within a canal of said subject.

2. The propelling device according to claim 1, wherein said second drive mechanism has a rotary body that rotates around an axis of said insertion part by the driving force received from said first drive mechanism in a state where said second drive mechanism comes into contact with an inner wall surface of said canal.

3. The propelling device according to claim 2,
wherein said first drive mechanism includes:
a driving-force transmission gear having a rotating shaft parallel to said axis, said driving-force transmission gear partially protruding from an opening formed at an outer periphery of said inserting part; and
a torque wire for transmitting power from said drive source to said rotating shaft so as to rotate said driving-force transmission gear, and
wherein said second drive mechanism includes:
an internal cylinder that is detachably mounted on the outer periphery of said insertion part, and has an opening for a gear from which said driving-force transmission gear is partially exposed;
a cylindrical worm gear that is rotatably attached to the outside of said internal cylinder, has a gear tooth portion which meshes with said driving-force transmission gear on an inner peripheral surface thereof, and is rotated by driving force received from said driving-force transmission gear; and
a rotary body drive gear that is provided outside said worm gear, and rotates said rotary body by the driving force received from said worm gear.

4. The propelling device according to claim 3, wherein said second drive mechanism further includes a housing cylinder that is detachably provided outside said worm gear to house said worm gear, and rotatably holds said rotary body drive gear.

5. The propelling device according to claim 2, wherein said first drive mechanism includes:
an internal cylinder provided inside said insertion part;
a cylindrical worm gear that is rotatably attached to the outside of said internal cylinder, said worm gear at least partially being exposed from an opening for a worm gear provided at an outer periphery of said insertion part;
a worm gear rotation driving device that receives the driving force from said drive source, and rotates said worm gear in a circumferential direction thereof; and
a rotary body drive gear that is rotatably attached to said opening for a worm gear, and rotates said rotary body by the driving force received from said worm gear.

6. The propelling device according to claim 2,
wherein said first drive mechanism includes:
an internal cylinder provided inside said insertion part;
a cylindrical worm gear that is rotatably attached to the outside of said internal cylinder, said worm gear at least partially being exposed from an opening for a worm gear provided at an outer periphery of said insertion part;
a worm gear rotation driving device that receives the driving force from said drive source, and rotates said worm gear in a circumferential direction thereof, and
wherein said second drive mechanism includes:
a rotary body drive gear for rotating said rotary body by the driving force received from said worm gear; and
a gear holding device that is detachably provided outside said insertion part and rotatably holds said rotary body drive gear.

7. The propelling device according to claim 5,
wherein a gear tooth portion is formed along the circumferential direction of an inner periphery of said worm gear, and
wherein said worm gear rotation driving device has a driving-force transmission gear that has a rotating shaft parallel to said axis, and meshes with said gear tooth portion, and a torque wire that transmits power from said drive source to the rotating shaft of said driving-force transmission gear, thereby rotating said driving-force transmission gear.

8. The propelling device according to claim 2, wherein said second drive mechanism has an external cylinder that allows said insertion part to be inserted therethrough and extends along said axis, and said rotary body is wound around said external cylinder and is supported by said external cylinder so as to circulate along said axis.

9. The propelling device according to claim 8, further comprising a plurality of supporting rollers that are rotatably attached to said external cylinder and come into contact with an inner peripheral surface of said rotary body so as to support said rotary body in a circulating manner,
wherein said rotary body drive gear drives said rotary body in a state where said rotary body is pinched between said rotary body drive gear and said plurality of supporting rollers.

10. The propelling device according to claim 8, wherein said rotary body is formed in the shape of a bag so as to cover said external cylinder over its entire circumference.

11. A self-propellable endoscope comprising:
an insertion part inserted into a subject;
an operation part for operating said insertion part; and
a propelling device according to claim 1.
